(19) United States
(12) Patent Application Publication
(10) Pub. No.: US 2018/0003716 A1
(43) Pub. Date: Jan. 4, 2018

(54) DEVICES FOR DETECTION OF AN ANALYTE IN URINE AND METHODS OF USING SAME

(71) Applicants: John P. Studdiford, Lakewood Ranch, FL (US); Mairead H. Studdiford, Lakewood Ranch, FL (US)

(72) Inventors: John P. Studdiford, Lakewood Ranch, FL (US); Mairead H. Studdiford, Lakewood Ranch, FL (US)

(21) Appl. No.: 15/350,506

(22) Filed: Nov. 14, 2016

Related U.S. Application Data
(60) Provisional application No. 62/356,429, filed on Jun. 29, 2016.

(51) Int. Cl. G01N 33/66; C12Q 1/54; G01N 21/78

(52) U.S. Cl. CPC G01N 33/66; G01N 21/78; C12Y 101/03004; C12Q 1/54; G01N 2333/904; G01N 2800/042

(57) ABSTRACT

Disclosed herein are devices for detecting presence and/or amount of an analyte, such as glucose, in a urine sample when diluted in a toilet bowl containing water and methods of using same. The disclosed devices eliminate the need to handle urine samples or a device that has been contacted with a urine sample, and can be conveniently disposed of by flushing into a sewage or septic system.

### CROSS-REFERENCE TO RELATED APPLICATIONS

This application claims the benefit of U.S. Provisional Application Ser. No. 62/356,429, filed on Jun. 29, 2016, which is herein incorporated by reference in its entirety.

### FIELD

This disclosure relates to devices and methods for measuring analytes in urine, particularly glucose.

### BACKGROUND

Diabetes is a worldwide epidemic that affects an increasing percentage of people with each passing year. The costs of managing patients with diabetes are high, including physician costs, hospitalization, medication, co-morbid conditions and long-term management for patients with complications. Diabetes is a multifactorial disease that includes defects such as poor insulin secretion by the pancreas, limited glucose uptake by muscle and fat, overproduction of gluconeogenesis by the liver, and increased glucose uptake in the kidney. Numerous medications have been developed to address various defects that contribute to diabetes, in order to achieve the overall goal of lowering blood glucose levels.

Standard methods for monitoring patients with diabetes and treatment efficacy typically include blood tests for glucose and/or glycated hemoglobin (HbA1c) levels. However, there remains a need for non-invasive and convenient devices and methods for measuring glucose levels and/or levels of other analytes present in the urine.

### SUMMARY

Disclosed herein are devices for detecting the presence (including amount) or absence of an analyte, such as glucose, in a urine sample and methods of using same. The disclosed devices eliminate the need to handle urine samples, or a device that has been contacted with a urine sample, and can be conveniently disposed of by flushing into a sewage or septic system.

The present disclosure provides methods of detecting glucose in urine of a subject, the method comprising: placing in a toilet bowl containing water a device that comprises an absorbent substrate having one or more reagents in an amount sufficient to react with glucose present in the toilet bowl containing water and urine and produce a visually detectable color change in a detection region of the device or a visually detectable color change in the water of the toilet bowl; collecting urine from the subject in the toilet bowl; waiting a predetermined period of time; and visually determining presence or absence of a color change in the detection region of the device or a visually detectable color change in the water of the toilet bowl, wherein glucose is determined to be present in the urine where the detection region changes color or the water of the toilet bowl changes color.

Additionally, the present disclosure provides methods of detecting glucose in urine of a subject, the method comprising: placing in a receptacle containing water a device that comprises an absorbent substrate having one or more reagents in an amount sufficient to react with glucose present in the receptacle containing water and urine and produce a visually detectable color change in a detection region of the device or a visually detectable color change in the water of the receptacle; collecting urine from the subject in the receptacle; waiting a predetermined period of time; and visually determining presence or absence of a color change in the detection region of the device or a visually detectable color change in the receptacle, wherein glucose is determined to be present in the urine where the detection region changes color or the water of the receptacle changes color.

In some embodiments of each or any of the above- or below-mentioned embodiments, the subject is taking a sodium glucose cotransporter 2 (SGLT2) inhibitor.

In some embodiments of each or any of the above- or below-mentioned embodiments, the subject is a healthy subject.

In some embodiments of each or any of the above- or below-mentioned embodiments, the healthy subject is pregnant.

In some embodiments of each or any of the above- or below-mentioned embodiments, the detection of glucose in the urine indicates that the subject has diabetes.

In some embodiments of each or any of the above- or below-mentioned embodiments, the detection of glucose in the urine indicates that the subject may have diabetes and should visit a healthcare provider for diagnosis.

In some embodiments of each or any of the above- or below-mentioned embodiments, collecting urine from the subject in the toilet bowl is achieved by the subject directly urinating into the toilet bowl.

In some embodiments of each or any of the above- or below-mentioned embodiments, the one or more reagents for detection of glucose comprise glucose oxidase, a peroxidase, and a chromogen.

In some embodiments of each or any of the above- or below-mentioned embodiments, the device is comprised of a biodegradable material.

In some embodiments of each or any of the above- or below-mentioned embodiments, the biodegradable material is paper.

In some embodiments of each or any of the above- or below-mentioned embodiments, the device is flushable.

In some embodiments of each or any of the above- or below-mentioned embodiments, the predetermined period of time is 15 seconds to 3 minutes.

In some embodiments of each or any of the above- or below-mentioned embodiments, the device further comprises a positive control region spatially separated from the detection region and comprising glucose and one or more reagents for detection of glucose.

In some embodiments of each or any of the above- or below-mentioned embodiments, the device is placed in the toilet bowl prior to collection of urine from the subject in the toilet bowl.

In some embodiments of each or any of the above- or below-mentioned embodiments, the device is placed in the toilet bowl after collection of urine from the subject in the toilet bowl.

The present disclosure also provides a device for detecting glucose in a toilet bowl containing water and urine, the device comprising: an absorbent substrate comprising a first detection region having one or more reagents in an amount sufficient to react with glucose present in the toilet bowl containing water and urine and produce a visually detectable color change in the first detection region or a visually detectable color change in the water of the toilet bowl.

Additionally, the present disclosure also provides a device for detecting glucose in a receptacle containing water and urine, the device comprising: an absorbent substrate comprising a first detection region having one or more reagents in an amount sufficient to react with glucose present in the receptacle containing water and urine and produce a visually detectable color change in the first detection region or a visually detectable color change in the water of the receptacle.

In some embodiments of each or any of the above- or below-mentioned embodiments, the device is comprised of a biodegradable material.

In some embodiments of each or any of the above- or below-mentioned embodiments, the biodegradable material is paper.

In some embodiments of each or any of the above- or below-mentioned embodiments, the paper is toilet paper.

In some embodiments of each or any of the above- or below-mentioned embodiments, the device is flushable.

The present disclosure also provides a device for detecting presence of an analyte of interest in a toilet bowl containing water and urine, comprising a substrate having one or more reagents for detection of a first analyte in an amount sufficient to react with the first analyte present in the toilet bowl containing water and urine and produce a visually detectable color change.

In some embodiments of each or any of the above- or below-mentioned embodiments, the substrate comprises a first detection region impregnated with the one or more reagents for detection of the first analyte.

In some embodiments of each or any of the above- or below-mentioned embodiments, the one or more reagents for detection of a first analyte react with the first analyte present in the toilet bowl containing water and urine to produce a visually detectable color change in the first detection region.

In some embodiments of each or any of the above- or below-mentioned embodiments, the one or more reagents for detection of a first analyte react with the first analyte present in the toilet bowl containing water and urine to produce a visually detectable color change in the toilet bowl containing water and urine (e.g., within the water and urine).

In some embodiments of each or any of the above- or below-mentioned embodiments, the substrate is an absorbent substrate.

In some embodiments of each or any of the above- or below-mentioned embodiments, the analyte of interest is glucose, bilirubin, ketone, blood, protein, urobilinogen, nitrite, leukocytes, pH, and/or specific gravity.

In some embodiments of each or any of the above- or below-mentioned embodiments, the device is flushable.

In some embodiments of each or any of the above- or below-mentioned embodiments, the first detection region comprises all or substantially all of the substrate.

In some embodiments of each or any of the above- or below-mentioned embodiments, the first detection region comprises 30-70% of the substrate.

In some embodiments of each or any of the above- or below-mentioned embodiments, the device further comprises a positive control region spatially separated from the first detection region and comprising the first analyte and one or more reagents for detection of the first analyte.

In some embodiments of each or any of the above- or below-mentioned embodiments, the device further comprises a second detection region with one or more reagents for detection of a second analyte of interest in an amount sufficient to react with the second analyte present in the toilet bowl containing water and urine and produce a visually detectable color change (e.g., the one or more reagents for detection of the second analyte react with the second analyte present in the toilet bowl containing water and urine to produce a visually detectable color change in the second detection region or the one or more reagents for detection of the second analyte react with the second analyte present in the toilet bowl containing water and urine to produce a visually detectable color change in the toilet bowl containing water and urine (e.g., within the water and urine)).

In some embodiments of each or any of the above- or below-mentioned embodiments, the second detection region comprises 30-70% or more of the substrate.

In some embodiments of each or any of the above- or below-mentioned embodiments, the device further comprises a positive control region spatially separated from the first detection region and the second detection region and comprising the second analyte and one or more reagents for detection of the second analyte.

In some embodiments of each or any of the above- or below-mentioned embodiments, the absorbent substrate is round, square, or rectangular.

In some embodiments of each or any of the above- or below-mentioned embodiments, the first detection region is round, square, or rectangular.

In some embodiments of each or any of the above- or below-mentioned embodiments, the first analyte is glucose and the one or more reagents for detection of glucose comprise glucose oxidase, a peroxidase, and a chromogen.

In some embodiments of each or any of the above- or below-mentioned embodiments, the absorbent substrate is a biodegradable material.

In some embodiments of each or any of the above- or below-mentioned embodiments, the absorbent substrate is a biodegradable material compatible with a sewer system or a septic system.

In some embodiments of each or any of the above- or below-mentioned embodiments, the absorbent substrate comprises paper.

The present disclosure also provides a roll of toilet paper comprising one or more of the devices disclosed herein.

The present disclosure also provides a solid reagent complex for detecting presence of an analyte of interest in the bowl of a toilet containing water and urine, comprising one or more reagents for detection of an analyte in an amount sufficient to react with the analyte present in the toilet bowl containing water and urine and produce a visually detectable color change, wherein the reagent complex is a solid water soluble composition, and wherein the analyte of interest comprises glucose, bilirubin, ketone, blood, protein, urobilinogen, nitrite, leukocytes, pH, or specific gravity.

The present disclosure also provides a solid reagent complex for detecting presence of an analyte of interest in a receptacle containing water and urine, comprising one or more reagents for detection of an analyte in an amount sufficient to react with the analyte present in the receptacle containing water and urine and produce a visually detectable color change, wherein the reagent complex is a solid water soluble composition, and wherein the analyte of interest comprises glucose, bilirubin, ketone, blood, protein, urobilinogen, nitrite, leukocytes, pH, or specific gravity.

The present disclosure also provides a liquid reagent complex for detecting presence of an analyte of interest in the bowl of a toilet containing water and urine, comprising one or more reagents for detection of an analyte in an amount sufficient to react with the analyte present in the toilet bowl containing water and urine and produce a visually detectable color change, wherein the analyte of interest comprises glucose, bilirubin, ketone, blood, protein, urobilinogen, nitrite, leukocytes, pH, or specific gravity.

The present disclosure also provides a liquid reagent complex for detecting presence of an analyte of interest in a receptacle containing water and urine, comprising one or more reagents for detection of an analyte in an amount sufficient to react with the analyte present in the receptacle containing water and urine and produce a visually detectable color change, wherein the analyte of interest comprises glucose, bilirubin, ketone, blood, protein, urobilinogen, nitrite, leukocytes, pH, or specific gravity.

The present disclosure also provides a method of detecting an analyte of interest in urine of a subject, comprising: placing in a toilet bowl containing water a device comprising a substrate comprising a detection region with one or more reagents for detection of the analyte in an amount sufficient to react with the analyte present in the toilet bowl containing water and urine; collecting urine from the subject in the toilet bowl; waiting a predetermined period of time; and visually determining presence or absence of a color change in the detection region of the device or in the water and urine present in the toilet bowl, wherein the presence of a color change in the detection region of the device or the presence of a color change in the water and urine present in the toilet bowl indicates that the analyte of interest is present in the urine.

The present disclosure also provides a method of detecting an analyte of interest in urine of a subject, comprising: placing in a receptacle containing water a device comprising a substrate comprising a detection region with one or more reagents for detection of the analyte in an amount sufficient to react with the analyte present in the receptacle containing water and urine; collecting urine from the subject in the receptacle; waiting a predetermined period of time; and visually determining presence or absence of a color change in the detection region of the device or in the water and urine present in the receptacle, wherein the presence of a color change in the detection region of the device or the presence of a color change in the water and urine present in the receptacle indicates that the analyte of interest is present in the urine.

In an embodiment, the analyte of interest is glucose, bilirubin, ketone, blood, protein, urobilinogen, nitrite, leukocytes, pH, and/or specific gravity.

The present disclosure also provides a method of detecting an analyte of interest in urine of a subject, comprising: collecting urine from the subject in a toilet bowl containing water; placing in the toilet bowl containing water a device comprising an substrate having a detection region with one or more reagents for detection of the analyte in an amount sufficient to react with the analyte present in the toilet bowl containing water and urine, wherein the analyte of interest comprises glucose, bilirubin, ketone, blood, protein, urobilinogen, nitrite, leukocytes, pH, or specific gravity; waiting a predetermined period of time; and visually determining presence or absence of a color change in the detection region of the device or in the water and urine present in the toilet bowl, wherein the presence of a color change in the detection region of the device or the presence of a color change in the water and urine present in the toilet bowl indicates that the analyte of interest is present in the urine.

The present disclosure also provides a method of detecting an analyte of interest in urine of a subject, comprising: collecting urine from the subject in a receptacle containing water; placing in the receptacle containing water a device comprising an substrate having a detection region with one or more reagents for detection of the analyte in an amount sufficient to react with the analyte present in the receptacle containing water and urine, wherein the analyte of interest comprises glucose, bilirubin, ketone, blood, protein, urobilinogen, nitrite, leukocytes, pH, or specific gravity; waiting a predetermined period of time; and visually determining presence or absence of a color change in the detection region of the device or in the water and urine present in the receptacle, wherein the presence of a color change in the detection region of the device or the presence of a color change in the water and urine present in the receptacle indicates that the analyte of interest is present in the urine.

In an embodiment, the device is placed in the toilet bowl after the collecting the subject's urine in the toilet bowl.

In an embodiment, the device is placed in the toilet bowl before collecting the subject's urine in the toilet bowl.

The present disclosure also provides a method of detecting one or more analytes of interest in urine, comprising: placing in a toilet bowl containing water the device disclosed herein; collecting urine from the subject in the toilet bowl containing water; waiting a predetermined period of time; and visually determining presence or absence of a color change in the detection region of the device or in the water and urine present in the toilet bowl, wherein the presence of a color change in the detection region of the device or the presence of a color change in the water and urine present in the toilet bowl indicates that the analyte of interest is present in the urine.

The present disclosure also provides a method of detecting one or more analytes of interest in urine, comprising: placing in a receptacle containing water the device disclosed herein; collecting urine from the subject in the receptacle containing water; waiting a predetermined period of time; and visually determining presence or absence of a color change in the detection region of the device or in the water and urine present in the receptacle, wherein the presence of a color change in the detection region of the device or the presence of a color change in the water and urine present in the receptacle indicates that the analyte of interest is present in the urine.

The present disclosure also provides a method of detecting one or more analytes of interest in urine, comprising: collecting urine from a subject in a toilet bowl containing water; placing in the toilet bowl containing water the device disclosed herein; waiting a predetermined period of time; and visually determining presence or absence of a color change in the detection region of the device or the presence of a color change in the water and urine present in the toilet bowl, wherein the presence of a color change in the detection region of the device or the presence of a color change in the water and urine present in the toilet bowl indicates that the analyte of interest is present in the urine.

The present disclosure also provides a method of detecting one or more analytes of interest in urine, comprising: collecting urine from a subject in a receptacle containing water; placing in the receptacle containing water the device disclosed herein; waiting a predetermined period of time; and visually determining presence or absence of a color change in the detection region of the device or the presence of a color change in the water and urine present in the receptacle, wherein the presence of a color change in the detection region of the device or the presence of a color change in the water and urine present in the receptacle indicates that the analyte of interest is present in the urine.

In some embodiments of each or any of the above- or below-mentioned embodiments, the predetermined period of time is 15 seconds to 3 minutes.

In some embodiments of each or any of the above- or below-mentioned embodiments, the device comprises a positive control region and further comprising visually determining presence or absence of a color change in the positive control region, wherein presence of a color change in the positive control region indicates that the test is working properly.

The present disclosure also provides a method of detecting one or more analytes of interest in urine, comprising: placing in a toilet bowl containing water the reagent complex disclosed herein; collecting urine from a subject in the toilet bowl containing water; waiting a predetermined period of time; and visually determining presence or absence of a color change in the detection region of the device or the presence of a color change in the water and urine present in the toilet bowl, wherein the presence of a color change in the detection region of the device or the presence of a color change in the water and urine present in the toilet bowl indicates that the analyte of interest is present in the urine.

The present disclosure also provides a method of detecting one or more analytes of interest in urine, comprising: placing in a receptacle containing water the reagent complex disclosed herein; collecting urine from a subject in the receptacle containing water; waiting a predetermined period of time; and visually determining presence or absence of a color change in the detection region of the device or the presence of a color change in the water and urine present in the receptacle, wherein the presence of a color change in the detection region of the device or the presence of a color change in the water and urine present in the receptacle indicates that the analyte of interest is present in the urine.

The present disclosure also provides a method of detecting one or more analytes of interest in urine, comprising: collecting urine from a subject in a toilet bowl containing water; placing in the toilet bowl containing water the reagent complex disclosed herein; waiting a predetermined period of time; and visually determining presence or absence of a color change in the detection region of the device or a color change in the water and urine present in the toilet bowl, wherein the presence of a color change in the detection region of the device or the presence of a color change in the water and urine present in the toilet bowl indicates that the analyte of interest is present in the urine.

The present disclosure also provides a method of detecting one or more analytes of interest in urine, comprising: collecting urine from a subject in a receptacle containing water; placing in the receptacle containing water the reagent complex disclosed herein; waiting a predetermined period of time; and visually determining presence or absence of a color change in the detection region of the device or a color change in the water and urine present in the receptacle, wherein the presence of a color change in the detection region of the device or the presence of a color change in the water and urine present in the receptacle indicates that the analyte of interest is present in the urine.

In some embodiments of each or any of the above- or below-mentioned embodiments, the analyte of interest is glucose and the subject is diabetic.

In some embodiments of each or any of the above- or below-mentioned embodiments, the subject is taking a sodium glucose cotransporter 2 (SGLT2) inhibitor.

In some embodiments of each or any of the above- or below-mentioned embodiments, the subject is a healthy subject.

In some embodiments of each or any of the above- or below-mentioned embodiments, the healthy subject is pregnant.

In some embodiments of each or any of the above- or below-mentioned embodiments, the analyte of interest is glucose and a positive result indicates that the subject has diabetes.

In some embodiments of each or any of the above- or below-mentioned embodiments, the detection of glucose in the urine indicates that the subject may have diabetes and should visit a healthcare provider for an accurate diagnosis.

In some embodiments of each or any of the above- or below-mentioned embodiments, the analyte of interest is glucose and the detection of color change in the detection region of the device or a color change in the toilet bowl containing water and urine indicate that the subject has or may have diabetes and should visit a healthcare provider for diagnosis.

The present disclosure also provides a kit comprising one or more devices or reagent complexes as disclosed herein and instructions for use.

In some embodiments, disclosed herein are flushable devices for detecting presence of an analyte (such as glucose) in the bowl of a toilet containing water and urine that include an absorbent substrate including a detection region impregnated with one or more reagents for detection of an analyte in an amount sufficient to react with the analyte present in the toilet bowl containing water and urine and produce a visually detectable color change. In some examples, all or substantially all of the device is impregnated with the detection reagents. In other examples, only a portion of the device (such as 90% or less of the device) is impregnated with the analyte detection reagents and the device optionally further includes a spatially distinct positive control region including one or more reagents for detection of glucose or another analyte of interest. In a further embodiment, the device includes two or more regions impregnated with reagents for detection of different analytes of interest. The detection reagents may be chosen such that they produce different colors in response to presence of the analyte, allowing the user to distinguish the analyte(s) present in their urine.

In other embodiments, the detection reagents are in the form of a solid reagent complex (such as a dissolvable tablet or cake) containing reagents for analyte detection. In some examples, the reagent complex is a single-use composition (such as a single-use dissolvable tablet) or is a multi-use composition (such as a slowly dissolving solid tablet). In still further examples, the reagent complex is in a concentrated liquid form that can be dispensed into the toilet tank.

In some embodiments, a subject places in a toilet bowl containing water a device including an absorbent substrate having a detection region impregnated with one or more reagents for detection of an analyte in an amount sufficient to react with the analyte present in the toilet bowl containing water and urine. In some embodiments, the subject urinates in the toilet bowl containing water and waits for a predetermined period of time, then visually determines whether there is a color change in the detection region. The device may be disposed of by flushing the toilet.

Detecting a color change in the detection region of the device indicates presence of the analyte in the urine (for example, presence of the analyte in an amount above a pre-determined threshold level such as 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more than the amount of analyte detected in the urine of a normal subject (i.e., a subject without the disease or disorder)). The subject can record the results of the test (for example, presence or absence of color change), for example, to monitor effectiveness of a treatment (such as a SGLT2 inhibitor in a diabetic subject), and in some examples, communicates the test results to a health care provider.

In some embodiments of each or any of the above-embodiments, the device is not an assay test strip or does not comprise an assay test strip.

The foregoing and other features of the disclosure will become more apparent from the following detailed description, which proceeds with reference to the accompanying figures.

### BRIEF DESCRIPTION OF THE DRAWINGS

The foregoing summary as well as the following detailed description will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIGS. 1A and 1B are views of embodiments of a biodegradable urine analyte test device, where substantially all of the device is impregnated with detection reagents.

FIG. 2 is a view of an embodiment of a biodegradable urine test device with approximately half of the substrate impregnated with detection reagents.

FIGS. 3A and 3B are views of alternative embodiments of a biodegradable urine analyte test device including a portion impregnated with detection reagents and a positive control region.

FIG. 4 is a view of an embodiment of a biodegradable urine analyte test device for detection of two different analytes.

FIGS. 5A and 5B are perspective views of embodiments of the disclosure with a biodegradable urine analyte test incorporated into a roll of toilet paper.

### DETAILED DESCRIPTION

Medications have been developed to address various aspects of diabetes to lower blood glucose. These medications include insulin, biguanides (such as metformin), dipeptidyl peptidase-4 (DPP4) inhibitors (such as sitagliptin, saxagliptin, linagliptin, and alogliptin), sulfonylureas (such as glimepiride, glipizide, and glyburide), thiazolidinediones (such as pioglitazone, rosiglitazone, and lobeglitazone), glucagon-like peptide-1 (GLP-1) receptor agonists (such as liraglutide, exenatide, and albiglutide), and sodium glucose cotransporter 2 (SGLT2) inhibitors (such as canagliflozin, dapagliflozin, and empagliflozin). All of these medications, except for the SGLT2 inhibitors, reduce blood glucose through mechanisms independent of the kidney, with the goal of reducing blood glucose to target laboratory levels of HbA1c below 7%. HbA1c levels are an indicator of blood glucose levels over time, rather at a single point in time. The effectiveness and safety of these medications has been well proven, and although not all subjects reduce HbA1c to goal, they generally eliminate glucose spillover from the kidney. As a result there is typically little or no glucose in the urine for subjects taking these classes of medications, except for the SGLT2 inhibitors.

SGLT2 inhibitors are unique based on their direct mechanism of lowering blood glucose by reducing the uptake of glucose in the proximal tubule in the kidney (e.g., by inhibiting sodium glucose transport). This unique mechanism is at times confusing to physicians and subjects because historically glucose in the urine was seen as a negative and proved a subject had uncontrolled diabetes. With the use of SGLT2 inhibitors, the presence of glucose in the urine is both necessary and positive because it proves the medication is working by excreting unwanted sugar in the urine—resulting in lower blood glucose and weight decrease in subjects taking these medications. For example, dapagliflozin was shown in clinical studies to eliminate about 70-100 grams of sugar each day through the urine. It has also been established through long-term clinical trials (>2 years) that the mechanism of the SGLT2 drugs is constant, regardless of duration on drug.

Due to the direct mechanism of action on the kidney and corresponding glucose excretion in the urine, the ability to monitor glucose excretion in the urine, as well as to easily show health care providers and subjects that their prescribed SGLT2 inhibitor is working is both motivating and a positive reinforcement tool to enhance adherence and compliance. Thus disclosed herein are easy to use devices for detecting the presence or absence of glucose in a urine sample, for example by detecting presence of a color change on the device when in a toilet bowl containing water and urine. The ability to actively see that their medication is working, through a simple color changing glucose test on urination, can be highly beneficial to addressing improved drug regimen compliance, and thus management and control of diabetes by subjects.

In addition, over a million people are newly diagnosed with diabetes in the United States every year and around 27% of people with diabetes are undiagnosed (National Diabetes Statistics Report, Centers for Disease Control and Prevention, 2014). Gestational diabetes affects around 5-10% of pregnant women. Gestational diabetes places the mother at risk for complications during pregnancy and delivery and places both the mother at child at increased risk for diabetes later in life. Thus, the disclosed devices can also be used as an “early warning” system to alert apparently healthy individuals (e.g., individuals that have not previously been diagnosed by a healthcare provider as having diabetes) that they have glucose in their urine and should see their doctor for a health exam and quantitative glucose testing.

The present disclosure provides methods for detecting the presence or absence of glucose in a urine sample from a subject with a disease or disorder such as diabetes that is or has taken a SGLT2 inhibitor. Such methods may include placing one or more disclosed test devices having one or more glucose detection reagents such as glucose oxidase detection system reagents in a receptacle for a urine sample (e.g., a toilet bowl), collecting urine from the subject in the receptacle such as a toilet bowl (e.g., by the subject urinating into the receptacle (e.g., a toilet bowl), waiting a predetermined period of time (e.g., a few seconds to 1-2 minutes, such as about 30 seconds to 1 minute); and detecting the presence or absence of a color change in a detection region of the device or in the water and urine present in the receptacle (e.g., a toilet bowl), wherein the presence of a color change in the detection region of the device or in the water and urine present in the receptacle (e.g., a toilet bowl) indicates that the subject has glucose in their urine. Detection of a color change in the detection region or in the water in the receptacle (e.g., a toilet bowl) indicates presence of glucose in the urine. In an embodiment, in the presence of glucose in the urine above a threshold amount (e.g., 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more than the amount of glucose detected in the urine of a normal subject (i.e., a subject that has not or is not taking a SGLT2 inhibitor, or a normal subject without the disease or disorder such as diabetes). In an embodiment the color change may be from colorless (e.g., white) to a color such as red, orange, yellow, green, blue, indigo, or violet, or any variation thereof. The color change reaction may be quantitative and/or qualitative. Thus, it may indicate not only the presence of glucose, but may also indicate, by the intensity of the color change, the concentration (e.g., amount) of glucose in the urine.

The present disclosure provides methods of detecting glucose in urine of a subject, the method comprising: placing in a receptacle (e.g., a toilet bowl)containing water a device that comprises a substrate (e.g., an absorbent substrate) having one or more reagents in an amount sufficient to react with glucose present in the receptacle (e.g., a toilet bowl) containing water and urine and produce a visually detectable color change in a detection region of the device or a visually detectable color change in the water present in the receptacle such as a toilet bowl; collecting urine from the subject in the receptacle (e.g., a toilet bowl); waiting a predetermined period of time; and visually determining presence or absence of a color change in the detection region of the device or a visually detectable color change in the water of the receptacle (e.g., a toilet bowl), wherein glucose is determined to be present in the urine where the detection region changes color or the water of the receptacle (e.g., a toilet bowl) changes color. The detection of glucose in the urine indicates that the subject has or may have diabetes and should visit a healthcare provider for diagnosis.

In some embodiments, the subject may be a healthy subject. The healthy subject may be pregnant.

The device may be comprised of a biodegradable material and/or may be flushable.

The present disclosure also provides a device for detecting glucose in a toilet bowl containing water and urine, the device comprising: an absorbent substrate comprising a first detection region having one or more reagents in an amount sufficient to react with glucose present in the toilet bowl containing water and urine and produce a visually detectable color change in the first detection region or a visually detectable color change in the water of the toilet bowl.

### I. Device Embodiments

Disclosed herein are devices for measuring analyte levels in a urine sample including a diluted urine sample. The disclosed devices may be directly placed into any receptacle that contains or is capable of containing a urine sample. For example, the devices can be placed directly in a toilet bowl containing water prior to or after collection of urine in the toilet bowl (e.g., by a subject urinating into the toilet bowl). The device may be placed into the toilet bowl 10 seconds, 20 seconds, 30 seconds, 40 seconds, 50 seconds, 1 minute, 2 minutes, 3 minutes, 4 minutes, 5 minutes, 6 minutes, 7 minutes, 8 minutes, 9 minutes, 10 minutes, or more than 10 minutes, etc., prior to or after collection of urine in the toilet bowl).

Also provided are devices for detecting glucose in a toilet bowl containing water and urine, the device comprising: an absorbent substrate comprising a first detection region having one or more reagents in an amount sufficient to react with glucose present in the toilet bowl containing water and urine and produce a visually detectable color change in the first detection region or a visually detectable color change in the water of the toilet bowl, wherein the presence of a color change in the detection region of the device or the presence of a color change in the water and urine present in the toilet bowl indicates that the analyte of interest is present in the urine.

As used herein, “analyte” refers to a substance, compound, chemical constituent, or chemical property that is of interest, for example in a biological system or sample. Exemplary analytes include glucose, proteins, ketones, hemoglobin, bilirubin, urobilinogen, acetone, or nitrite. The disclosed devices can also be used to determine physical properties of a solution, such as pH or specific gravity, which are also referred to herein as “analytes.”

As used herein, “absorbent substrate” refers to a material that can absorb aqueous solutions (such as water or urine), for example, a wettable material. In some examples, the absorbent substrate is substantially flat and may have a single layer or two or more layers. The absorbent substrates utilized in the devices disclosed herein are flushable, for example, are materials that can acceptably be disposed of in a sewage or septic system.

A “subject” is a living vertebrate organism, a category that include both human and veterinary subjects, including human and non-human mammals. A “healthy” subject refers to a subject who has not been diagnosed with a particular disease or condition. This includes subjects who have a particular disease or condition, but do not recognize that they have the condition or have not yet been diagnosed with the condition. Thus, in the context of testing for glucose in the urine, a healthy subject can be a subject who does not have diabetes or a subject with undiagnosed diabetes. The subject may be a human patient.

As used herein, “diabetes” refers to diabetes mellitus, a disease caused by a relative or absolute lack of insulin leading to uncontrolled carbohydrate metabolism. Type 1 diabetes (sometimes referred to as “insulin-dependent diabetes” or “juvenile-onset diabetes”) is an auto-immune disease characterized by destruction of the pancreatic β cells that leads to a total or near total lack of insulin. In type 2 diabetes (T2DM); sometimes referred to as “non-insulin-dependent diabetes” or “adult-onset diabetes”), the body does not respond to insulin, though it is present.

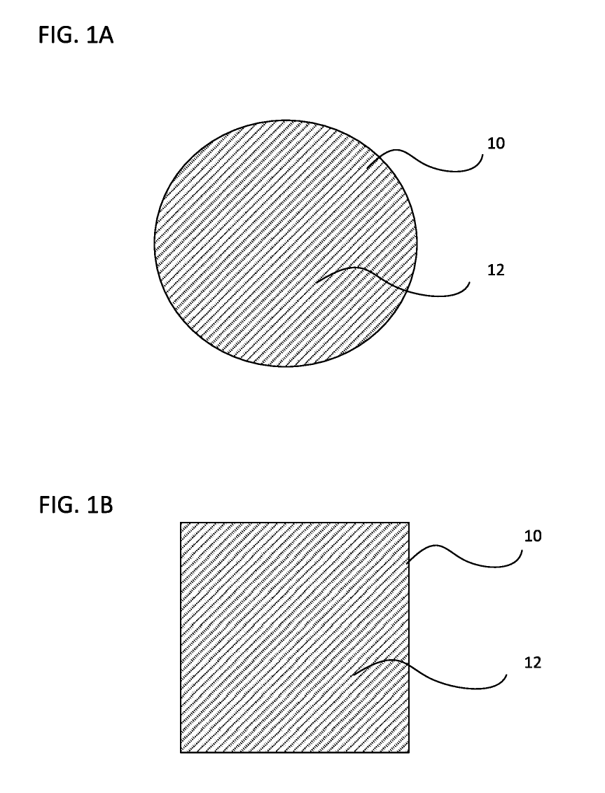

Referring to FIGS. 1A and 1B, in some embodiments, the device includes a substrate (e.g., an absorbent substrate) 10 having one or more reagents 12 for detection of an analyte (such as glucose). In the presence of the analyte, a colored compound is formed. The substrate 10 is of a convenient size and shape for handling, such as circular (FIG. 1A), square (FIG. 1B), or rectangular. The size of the substrate may be about 2-6 inches in diameter (for example, 3-5 inches, 4-6 inches or 3-4 inches in diameter) or about 4-25 square inches (for example about 4, 5, 9, 10,5, 16, 18, or 25 square inches). In one embodiment, the substrate is the size and shape of a sheet of toilet paper. The size and shape of the substrate 10 should be sufficiently large for a user to easily read the result, but still be flushable in conventional household toilets. Suitable biodegradable and flushable absorbent substrates include, but are not limited to paper, cellulose, polyester, natural fibers, or a combination thereof. The substrate should be compatible with standard sewer and septic systems. In some examples, the substrate has more than one layer (for example, the substrate is two-ply or three-ply); however, single layer (single-ply) substrates are discussed herein as exemplary embodiments. Preferably, the device is not an assay test strip.

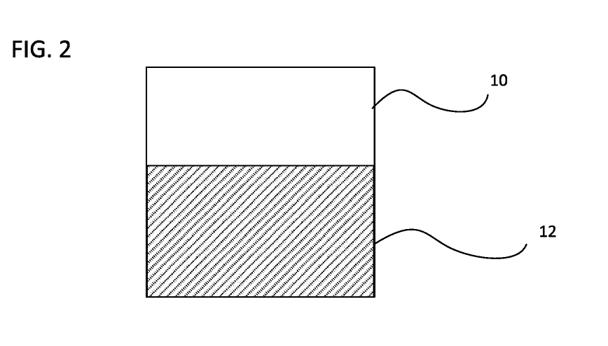

The detection region 12 can be any size and shape, including round, square, oval, or other shape, and of a sufficiently large area for a user to easily read the result. In some embodiments, all or substantially all of the substrate may be impregnated with reagents for detection of an analyte, such as glucose (e.g., FIGS. 1A and 1B), thus, the detection region 12 is all or substantially all of the area of the substrate 10. In other embodiments, only a portion of the substrate is impregnated with the analyte detection reagents. Referring to FIG. 2, the detection region 12 is approximately half of the substrate 10 area. In other examples, the detection region 12 can be included in about 20-100% of the area of the substrate 10 (such as about 25-50%, 30-70%, 40-80%, or 50-100% of the area). While the embodiments shown in FIGS. 1-5 illustrate generally round, square, or rectangular detection regions, the detection region can be of any desired shape, determined by the placement of the detection reagents on the substrate. For example, the detection region can be in the shape of a plus sign or another symbol(s) or can include words (such as “positive,” “high glucose,” or “see your doctor”).

The one or more detection reagents (such as a chromogenic detection system, discussed below) in the detection region 12 are present in an amount sufficient to detect the analyte of interest in a dilute sample, such as a urine sample diluted in the water in a toilet bowl.

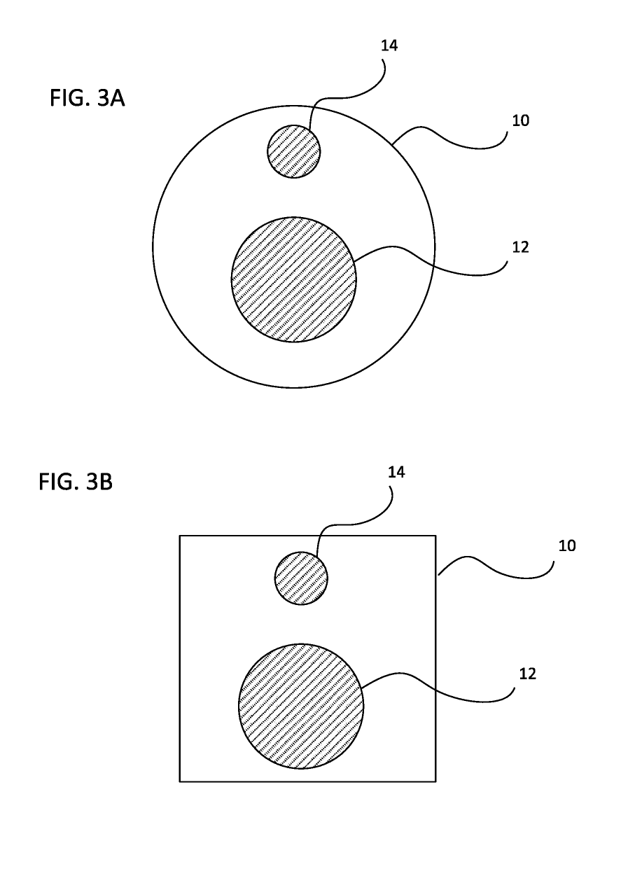

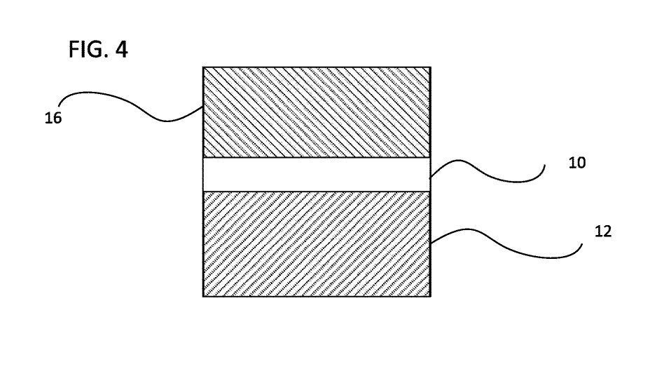

Referring to FIGS. 3A and 3B, in some embodiments, the device also includes a positive control indicator, such as a positive control region 14 impregnated with one or more reagents for detection of an analyte and the analyte to be detected, such that when the absorbent material is contacted with water or an aqueous solution, a reaction producing a colored compound occurs. The positive control region 14 is spatially separated from and distinguishable from the detection region, for example by having a different size and/or shape than the detection region 12. In alternate embodiments, the positive control region 14 impregnated with one or more reagents for detection of an analyte and the analyte to be detected is present within a urine sample.

In additional embodiments, the devices disclosed herein include reagents for detection of two or more (such as 2, 3, 4, or more) different analytes. FIG. 4 illustrates an exemplary embodiment of a device with an absorbent substrate 10 having a detection region for a first analyte 12 and a detection region for a second analyte 16. The detection reagents are selected to produce different colors or patterns for each analyte being detected, to allow the user to determine which analyte(s) are positive in a urine sample. In some examples (such as that shown in FIG. 4), the two or more detection regions are spatially separated on the substrate. In some examples, the devices also optionally include two or more positive control regions. For example, in embodiments where the device includes two or more detection regions, the device can also include distinct positive control regions for each analyte detected by the different detection regions.

The device can be individually wrapped or packaged, for example in a pouch or other sealed container to prevent contamination of the device prior to use. In other examples, the device is placed in packages of two or more, such as 10-500 (for example, 10-50, 20-75, 50-100, 100-300, 200-400, or 300-500 per package). In some examples, two or more devices can be packaged in a dispenser and an individual device (sheet) can be removed and placed in a toilet bowl when ready for use.

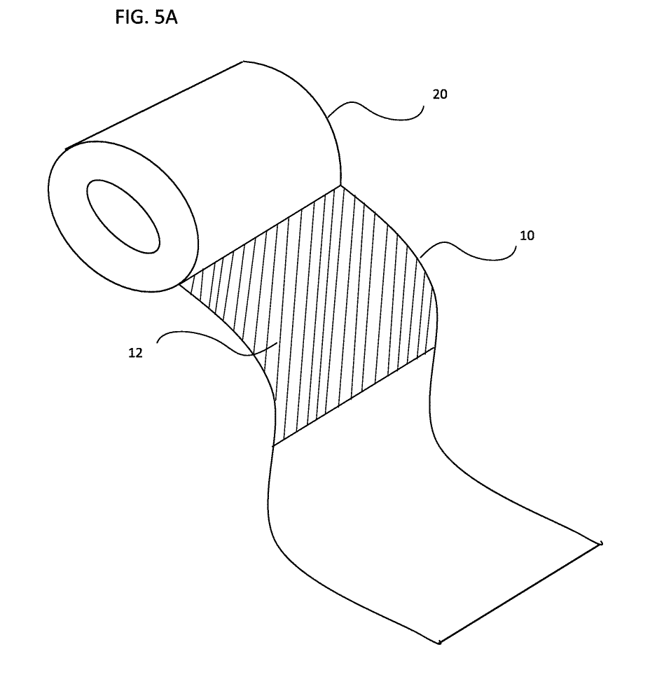

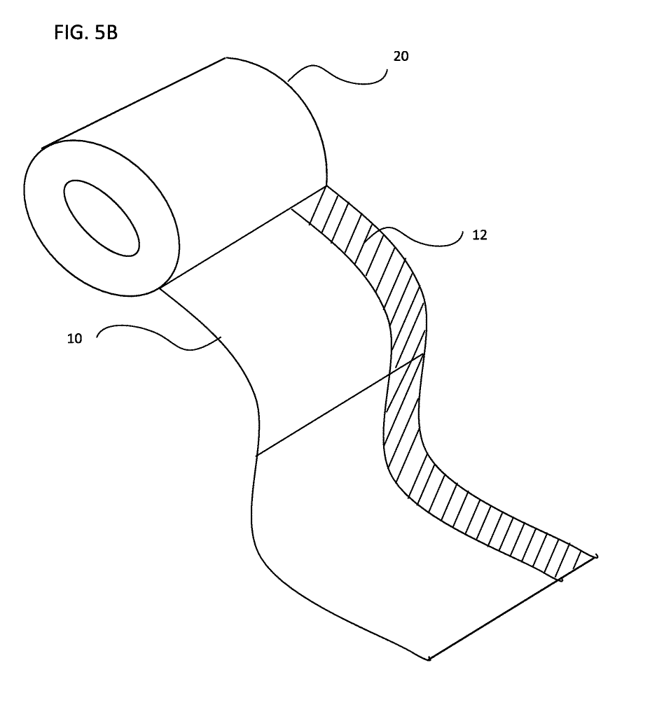

In another example, the device is incorporated into a roll of toilet paper. Referring to FIG. 5A at least one sheet of a roll of toilet paper 20 is an absorbent substrate 10 with detection region 12 including reagents for detecting an analyte, such as glucose. In some examples, one sheet out of every 2, 5, 10, 15, or 20 sheets of toilet paper in the roll is a disclosed device (e.g., an absorbent substrate impregnated with reagents for detecting an analyte in a urine sample when diluted in a toilet bowl). FIG. 5A illustrates an embodiment where an entire sheet of toilet paper is impregnated with detection reagents. In another embodiment, illustrated in FIG. 5B, each sheet of the roll of toilet paper 20 is an absorbent substrate 10 with a portion being the detection region 12 including reagents for detecting an analyte. FIG. 5B shows a strip each sheet of toilet paper impregnated with detection reagents; however, other configurations, such as other portions of the toilet paper sheet impregnated with detection reagents or optionally also including a positive control region are also contemplated. In another embodiment, the device is incorporated into a cleansing cloth including, for example, a flushable cleansing cloth.

In yet further embodiments, the detection reagents are in the form of a reagent complex (such as a dissolvable tablet or cake) containing reagents for analyte detection. In addition to detection reagents (discussed below), the reagent complex may also include additional components, such as binders, preservatives, fragrance, or other non-reactive components. In some examples, the reagent complex is a water soluble single-use solid tablet including reagents for detecting an analyte. In other examples, the reagent complex is a slowly dissolving solid tablet (for example, a urinal cake or a solid tablet that can be placed in a toilet tank) that includes reagents sufficient for multiple uses. In still further examples, the reagent complex is in a concentrated liquid form that can be dispensed into the toilet tank by an automatic dispenser. The reagent complex may comprise one or more reagents in an amount sufficient to react with glucose present in the toilet bowl containing water and urine and produce a visually detectable color change in the water and urine present in the toilet bowl, wherein the presence of a color change in the water and urine present in the toilet bowl indicates that the analyte of interest is present in the urine. In an embodiment, the reagent complex is incorporated into a roll of toilet paper. In some examples, one sheet out of every 2, 5, 10, 15, or 20 sheets of toilet paper in the roll comprises a reagent complex. In another embodiment, each sheet of the roll of toilet paper comprises a reagent complex. In another embodiment, the reagent complex is incorporated into a cleansing cloth including, for example, a flushable cleansing cloth.

In particular examples, the reagents for detection of an analyte (such as glucose) are present on the device (such as in the detection region) or in the toilet bowl (in the case of reagents added directly to the toilet bowl) in an amount sufficient to react with the analyte present in the toilet bowl containing water and urine and produce a visually detectable color change in the detection region of the device or in the water and urine within the toilet bowl.

Urine void volumes are assumed to be from about 30 ml to 400 ml and toilet bowl water volumes are assumed to be from about 3 liters to about 8 liters. Thus, in some examples, the reagents are present in an amount capable of detecting the analyte in urine that is diluted by about 5-fold to about 300-fold (such as about 10-fold to 50-fold, about 20-fold to 100-fold, about 50-fold to 200-fold, or about 200-fold to 300-fold). In one non-limiting example, the disclosed devices are capable of detecting glucose present in an undiluted urine sample at an amount of 50 mg/dl or more. Thus, in some examples, the disclosed devices or reagent complexes can detect glucose in a diluted urine sample in a toilet bowl at a concentration of at least about 0.1 mg/dl (for example, at least about 0.2 mg/dl, 0.3 mg/dl, 0.4 mg/dl, 0.5 mg/dl, 0.6 mg/dl, 0.7 mg/dl, 0.8 mg/dl, 0.9 mg/dl, 1 mg/dl, 1.25 mg/dl, 1.5 mg/dl, 1.75 mg/dl, 2 mg/dl, 2.5 mg/dl, 3 mg/dl, 4, mg/dl, 5 mg/dl, 6 mg/dl, 7 mg/dl, 8 mg/dl, 9 mg/dl, 10 mg/dl, or more). In other examples, the disclosed devices or reagent complexes are capable of detecting additional analytes (such as ketones, protein, bilirubin, urobilinogen, or nitrite) at concentrations present in urine diluted by the amount of water present in a toilet bowl.

In some embodiments, the reagents for detection of an analyte are reagents that produce a colored product in the presence of the analyte. The detection reagents include one or more chromogens that can be converted to a colored product under appropriate conditions. In some exemplary detection systems, following an initial reaction of an analyte to produce one or more reaction products, the reaction product (for example, in the presence of one or more additional reagents) causes conversion of a chromogen to a colored product. In other examples, the analyte may react directly with a detection reagent to produce a colored product. Exemplary detection systems are discussed below; however, one of ordinary skill in the art can identify additional detection reagents that can be utilized for detection of an analyte of interest in urine.

For example, the detection reagent may be a glucose oxidase system for detection of glucose. The glucose oxidase system includes glucose oxidase, which catalyzes oxidation of glucose to form D-glucono-δ-lactone and hydrogen peroxide, and a chromogen, which reacts with the resulting hydrogen peroxide (e.g., mediated by a peroxidase) to form a colored product that is visually detectable. Chromogens that can be used in the device as part of a glucose oxidase system include potassium iodide, 3,3′,5,5′-tetramethylbenzidine (TMB), or 4-aminoantipyrine plus phenol (or a phenol substitute, such as pHBS). One of ordinary skill in the art can select additional suitable chromogens that can form a colored product on reaction with hydrogen peroxidase (including, but not limited to 3,3′-diaminobenzidine (DAB), 2-amino-4-hydroxybenzenesulfonic acid (AHBS), or 10-acetyl-3,7-dihdroxyphenoxzaine).

Exemplary detection systems for other analytes and their typical range of detection in undiluted urine are shown in Table 1. Additional detection reagents can be identified for these or other analytes of interest.

TABLE 1
Exemplary reagent detection systems for urine analytes

| | | Range of | |
|---|---|---|---|
| | | Detection in | |
| Analyte | Detection Reagent(s) | Undiluted Urine | |
| Bilirubin | 2,4-dichloroaniline diazonium salt; or | 25-150 | mg/dl |
| | 2,6-dichlorobenzen-diazonium- | | |
| | tetrafluoroborate; or 2,4-dichlorobenzene | | |
| | amine diazonium salt | | |
| Ketone | sodium nitroprusside | 0.2-1 | mg/dl |
| Specific | bromthymol blue and poly(methyl | | |
| Gravity | vinyl ether/maleic anhydride) | | |
| Blood | diisopropylbenzene dihydroperoixde and | 0.015-0.3 | mg/dl |
| | 3,3',5,5'-tetramethylbenzidine (TMB); or | hemoglobin | |
| | 2,5-dimethylhexane-2,5-dihydroperoxide | | |
| | and TMB; or | | |
| | cumene hydroperoxide and TMB; or | | |
| | cumene hydroperoxide and O-tolidine | | |
| pH | bromthymol blue and methyl red | | |
| Protein | tetrabromphenol blue; or | 5-30 | mg/dl |
| | 3’,3”,5’,5”-tetrachlorophenol-3,4,5,6- | | |
| | tetrabromsulfophthalein | | |
| Urobilinogen | p-diethylaminobenzaldehyde; or | 0.2-1 | mg/dl |
| | 4-methloxybenzene-diazonium- | | |
| | tetrafluoroborate; or | | |
| | Fast B blue | | |
| Nitrite | p-arsanilic acid and 1,2,3,4- | 0.05-0.1 | mg/dl |

| | | |
|---|---|---|
| | tetrahydrobenzo(h) quinolin-3-ol; or | |
| | p-arsanilic acid-N-1-(naphthol)- | |
| | ethylenediamine and tetrahydroquinoline; | |
| | or sulfanilamide and N-(naphthyl) | |
| | ethylenediammonium dihydrochloride; or | |
| | sulfanilamide and 3-hydroxy-1,2,3,4,- | |
| | tetrahydro-benzo (h) quinoline | |
| Leukocytes | pyrrole amino acid ester and | 5-40 white |
| | diazonium salt; or | blood cells/μl |
| | indoxylcarbonic acid ester | |
| | and diazonium salt; or | |
| | carboxylic acid ester and | |
| | diazonium salt; or phenylthiazole | |
| | amino acid ester and | |
| | diazonium salt | |

In some embodiments, one or more of the disclosed devices are included in a kit with instructions for use. In some examples, the kit includes one or more rolls of toilet paper that include one or more of the disclosed devices incorporated as sheets of toilet paper in the roll. In other examples, the kit includes one or more packages of the disclosed devices, such as a package of the disclosed devices in the form of sheets or wipes in a dispenser box that can be placed near a toilet, for example, along with the directions for use. In still further examples, the kit includes one or more packages of reagent complexes such as a package of reagent complexes in a dispenser box that can be placed near a toilet, for example, along with directions for use.

### II. Methods of Use

Disclosed herein are methods of determining the presence and/or amount of one or more analytes in a urine sample utilizing the disclosed analyte detection devices. The amount of the one or more analytes can be detected qualitatively, semi-quantitatively, or quantitatively. In some embodiments, the presence or absence of an analyte in a urine sample (either absolute presence or absence, or presence or absence relative to a cut-off level) is determined. Thus, the disclosed devices and methods can in some examples provide a simple yes/no determination of the presence of an analyte or presence of an analyte above a particular cut-off level. The particular cut-off level may be 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more than the amount of the analyte detected in the urine of a normal subject (i.e., a subject without a disease or disorder such as diabetes).

The present disclosure also provides methods of detecting glucose in urine of a subject, the method comprising: placing in a receptacle (e.g., a toilet bowl)containing water a device that comprises an absorbent substrate having one or more reagents in an amount sufficient to react with glucose present in the receptacle (e.g., a toilet bowl)containing water and urine and produce a visually detectable color change in a detection region of the device or a visually detectable color change in the water of the receptacle (e.g., a toilet bowl); collecting urine from the subject in the receptacle (e.g., a toilet bowl); waiting a predetermined period of time; and visually determining presence or absence of a color change in the detection region of the device or a visually detectable color change in the water of the receptacle (e.g., a toilet bowl), wherein glucose is determined to be present in the urine where the detection region changes color or the water of the receptacle (e.g., a toilet bowl) changes color.

In some embodiments, the disclosed device is placed in a receptacle (e.g., a toilet bowl) prior to voiding (urinating) by a subject. The subject may void either directly on the device or the device may be contacted with the urine indirectly in the toilet water. In other embodiments, the disclosed test device is placed in the receptacle (e.g., a toilet bowl)after voiding by the subject. The detection reagent(s) on the device react with the analyte in the urine and produce a colored product when the analyte is present in the urine. The subject or a healthcare practitioner observes the presence or absence of a colored product to determine whether the analyte of interest is present in the urine sample (for example, present above a threshold level). The threshold may be 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more than the amount of analyte detected in the urine of a normal subject (i.e., a subject without the disease or disorder)). After observing the result (such as the presence or absence of a color change) the device may be flushed along with the toilet water, disposing of the device in a sanitary manner.

If a positive control region is present on the device, the presence of a color change in the positive control region indicates that the test is working properly. If there is no color change in the positive control region, the test is considered to fail, and is discarded (e.g., flushed) and the test can be repeated with a new device.

In other embodiments, a disclosed reagent complex (such as a dissolvable single-use solid tablet) is placed in a toilet prior to voiding by a subject. The subject may void immediately, or wait for the reagent complex tablet to dissolve prior to voiding. In other embodiments, the reagent complex tablet is placed in the toilet after voiding by the subject. The detection reagent(s) react with the analyte in the urine and produce a colored product when the analyte is present in the urine. The subject observes the presence or absence of a colored product to determine whether the analyte of interest is present in the urine sample (for example, present above a threshold level). The threshold may be 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more than the amount of analyte detected in the urine of a normal subject (i.e., a subject without the disease or disorder)). After observing the result (such as the presence or absence of a color change) the reagents may be flushed along with the toilet water, disposing of the reagents in a sanitary manner.

In additional embodiments, a slow-release reagent complex tablet is placed in a receptacle (e.g., a toilet bowl including a toilet tank or urinal). In other examples, the detection reagents are automatically dispensed into the toilet bowl, for example, from a dispenser in the toilet tank. The detection reagents are thus present in the toilet bowl prior to voiding. The subject voids in the toilet bowl and observes the presence or absence of a colored product to determine whether the analyte of interest is present in the urine sample (for example, present above a threshold level). The threshold may be 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more than the amount of analyte detected in the urine of a normal subject (i.e., a subject without the disease or disorder)). After observing the result (such as the presence or absence of a color change) the used reagents may be flushed along with the toilet water, and fresh reagents are supplied in the clean water added to the toilet bowl following the flush.

In one specific example, the methods include determining the presence of glucose in a urine sample. The methods include placing a disclosed flushable test device including glucose detection reagents (such as glucose oxidase detection system reagents) in a toilet and voiding into the toilet by the subject. Following a sufficient period of time for the detection reagents to react (for example, a few seconds to 1-2 minutes, such as about 30 seconds to 1 minute), the subject observes the device to detect whether a color change occurs in the detection region of the device. Detection of a color change (such as presence of a colored compound) in the detection region indicates presence of glucose in the urine. In some examples, detecting a color change indicates presence of glucose above a particular threshold level.

In some examples, the subject is taking an SGLT2 inhibitor, and a positive result (e.g., presence of glucose in the urine) above a threshold level indicates that the treatment is working. In other examples, the subject is not taking an SGLT2 inhibitor and a positive result above a threshold level indicates that the subject has elevated blood glucose. In an unmedicated subject, a positive result above a threshold level may indicate that the subject has or may have diabetes and should visit a healthcare provider for diagnosis. In a subject taking one or more medications for diabetes other than an SGLT2 inhibitor, a positive result above a threshold level may indicate that their treatment is less than optimally effective and should be adjusted. The threshold level may be 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or more than the amount of analyte detected in the urine of a normal subject (i.e., a subject without the disease or disorder)).

In some embodiments, the subject records the result of the test, which may be performed a single time or periodically (for example, once per day, every other day, once per week, biweekly, or so on). The test results can be communicated to a health care provider immediately or on a periodic basis and can be used to adjust the subject's treatment plan.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method of detecting glucose in urine of a diabetic subject taking an amount of a sodium glucose cotransporter 2 (SGLT2) inhibitor, the method comprising:
placing in a toilet bowl containing water a device that comprises an absorbent substrate having one or more reagents in an amount sufficient to react with glucose present in the toilet bowl containing water and urine and produce a visually detectable color change in a detection region of the device or a visually detectable color change in the water of the toilet bowl;
collecting urine from the diabetic subject in the toilet bowl;
waiting a predetermined period of time; and
visually determining presence or absence of a color change in the detection region of the device or a visually detectable color change in the water of the toilet bowl to obtain a test result,
recording the test result;
communicating the test result to a health care provider; and
monitoring the effectiveness of the SGLT2 inhibitor based on the test result,
wherein glucose is determined to be present in the urine where the detection region changes color or the water of the toilet bowl changes color, wherein the presence of glucose above a threshold level indicates that the SGLT2 inhibitor is effective in the diabetic subject, wherein the device is paper, and wherein the device further comprises a positive control region spatially separated from the detection region and comprising glucose and one or more reagents for detection of glucose.

2-6. (canceled)

7. The method of claim 1, wherein collecting urine from the subject in the toilet bowl is achieved by the subject directly urinating into the toilet bowl.

8. The method of claim 1, wherein the one or more reagents for detection of glucose comprise glucose oxidase, a peroxidase, and a chromogen.

9. The method of claim 1, wherein the device is comprised of a biodegradable material.

10. (canceled)

11. The method of claim 1, wherein the device is flushable.

12. The method of claim 1, wherein the predetermined period of time is 15 seconds to 3 minutes.

13. (canceled)

14. The method of claim 1, wherein the device is placed in the toilet bowl prior to collection of urine from the subject in the toilet bowl.

15. The method of claim 1, wherein the device is placed in the toilet bowl after collection of urine from the subject in the toilet bowl.

16. A device for detecting glucose in a toilet bowl containing water and urine, the device comprising: an absorbent substrate comprising a first detection region having one or more reagents in an amount sufficient to react with glucose present in the toilet bowl containing water and urine and produce a visually detectable color change in the first detection region or a visually detectable color change in the water of the toilet bowl.

17. The device of claim 16, wherein the device is comprised of a biodegradable material.

18. The device of claim 16, wherein the biodegradable material is paper.

19. The device of claim 18, wherein the paper is toilet paper.

20. The method of claim 16, wherein the device is flushable.

21. The method of claim 1 further comprising administering the SGLT2 inhibitor to the subject.
